(19) United States
(12) Patent Application Publication
(10) Pub. No.: US 2015/0101986 A1
(43) Pub. Date: Apr. 16, 2015

(54) MIXED MATRIX POLYMERIC MEMBRANES

(71) Applicant: SABIC Global Technologies B.V., Amsterdam (NL)

(72) Inventors: Ihab Nizar ODEH, Sugar Land, TX (US); Yunyang LIU, Thuwal (SA)

(21) Appl. No.: 14/508,848

(22) Filed: Oct. 7, 2014

Related U.S. Application Data
(60) Provisional application No. 61/891,774, filed on Oct. 16, 2013.

(51) Int. Cl. B01D 53/22; B01D 53/04; B01D 53/047; B01D 61/36; C08G 73/10; B01D 63/06; B01D 63/08; B01D 63/10; C07C 7/144; B01D 71/64; B01D 63/02

(52) U.S. Cl. CPC B01D 53/228; B01D 71/64; B01D 53/0407; B01D 53/047; B01D 61/366; B01D 61/362; B01D 63/02; B01D 63/06; B01D 63/08; B01D 63/10; B01D 61/364; C07C 7/144; C08G 73/1067; B01D 2053/221; B01D 2053/224; B01D 2053/223; B01D 2253/204; B01D 2256/24; B01D 2257/702
USPC 210/640; 96/4; 96/14; 96/10; 95/45; 96/143; 95/96; 210/500.25; 210/500.23; 525/436

(57) ABSTRACT

Disclosed are mixed matrix polymeric membranes comprising a plurality of metal-organic frameworks (MOFs), or in some aspects a zeolitic imidazolate frameworks (ZIFs), and a polymeric matrix, wherein the plurality of MOFs are attached to the polymeric matrix through covalent or hydrogen bonds or Van der Waals interaction.

### CROSS REFERENCE TO RELATED APPLICATIONS

This application claims the benefit of U.S. Provisional Application No. 61/891,774, filed Oct. 16, 2013, the contents of which are incorporated into the present application by reference.

### BACKGROUND OF THE INVENTION

A. Field of the Invention

The present invention relates to mixed matrix polymeric membranes in which metal-organic frameworks (MOFs) are attached to the polymers such that voids between the interface of the MOFs and polymers are reduced in number or size or both. In particular embodiments, the attachment is through the formation of a covalent bond between the MOFs and polymers. This results in mixed matrix membranes having improved selectivity parameters.

B. Description of Related Art

A membrane is a structure that has the ability to separate one or more materials from a liquid, vapour or gas. It acts like a selective barrier by allowing some material to pass through (i.e., the permeate or permeate stream) while preventing others from passing through (i.e., the retentate or retentate stream). This separation property has wide applicability in both the laboratory and industrial settings in instances where it is desired to separate materials from one another (e.g., removal of nitrogen or oxygen from air, separation of hydrogen from gases like nitrogen and methane, recovery of hydrogen from product streams of ammonia plants, recovery of hydrogen in oil refinery processes, separation of methane from the other components of biogas, enrichment of air by oxygen for medical or metallurgical purposes, enrichment of ullage or headspace by nitrogen in inerting systems designed to prevent fuel tank explosions, removal of water vapor from natural gas and other gases, removal of carbon dioxide from natural gas, removal of H₂S from natural gas, removal of volatile organic liquids (VOL) from air of exhaust streams, desiccation or dehumidification of air, etc.).

Examples of membranes include polymeric membranes such as those made from polymers, liquid membranes (e.g., emulsion liquid membranes, immobilized (supported) liquid membranes, molten salts, etc.), and ceramic membranes made from inorganic materials such as alumina, titanium dioxide, zirconia oxides, glassy materials, etc.

For gas separation applications, the membrane of choice is typically a polymeric membrane. One of the issues facing polymeric membranes, however, is their well-known trade-off between permeability and selectivity as illustrated by Robeson's upper bound curves (Robeson, 1991; Robeson, 2008). In particular, there is an upper bound for selectivity of, for example, one gas over another, such that the selectivity decreases with an increase in membrane permeability.

Metal-organic frameworks (MOFs) such as zeolitic imidazolate frameworks (ZIFs) have been previously incorporated into polymeric membranes to create mixed matrix membranes. The use of MOFs were to increase the permeability of said membranes. These mixed matrix membranes were prepared by blending ZIFs with polymers, in which no chemical reaction between the ZIFs and the polymers occurred. This allowed for an increase in the permeability of the membranes, due to the poor interaction between the MOFs and polymers at the polymer-zeolite interface. In particular, non-selective interfacial voids were introduced in the membranes such that the voids allowed for increased permeability but decreased selectivity of given materials. This has been referred to as a “sieve-in-a-cage” morphology (Hillock et al., 2008). FIGS. 1A-B illustrate prior art membranes exhibiting “sieve in a cage” morphology (Mahajan, et al., 2002).

Such “sieve-in-a-cage” morphology has resulted in mixed matrix membranes that fail to perform above a given Roberson upper bound trade-off curve. That is, a majority of such membranes fail to surpass the permeability-selectivity tradeoff limitations, thereby making them less efficient and more costly to use. As a result, additional processing steps may be required to obtain the level of gas separation or purity level desired for a given gas.

### SUMMARY OF THE INVENTION

A solution to the disadvantages of the currently available mixed matrix membranes has been discovered. In particular, the solution is based on a surprising discovery that the attachment of metal-organic frameworks (MOFs) to polymers of a polymeric membrane through a chemical bond such as a covalent bond can increase the selectivity of the membrane. Without wishing to be bound by theory, it is believed that such an attachment between the MOFs and polymers reduces the size and number of interfacial voids between said MOFs and polymers, thereby reducing or avoiding the aforementioned “sieve-in-a-cage” morphology. This can result in an increase in the selectivity of the membrane for given materials. This selectivity can be further tuned by modifying the number of chemical bonds between said MOFs and polymers such that particular membranes can be obtained for particular applications (e.g., separation of olefins from paraffins, carbon dioxide from natural gas, hydrogen gas from carbon monoxide, etc.). Additionally, it is also believed that the membranes of the present invention can be further tuned by modifying the pore sizes of the MOFs. In this regard, the chemical bonds between the MOFs and polymers help to reduce or prevent the “sieve-in-a-cage” morphology, while the pore sizes of the MOFs can be used to further select which materials permeate through the pores of the MOFs and which materials do not permeate through said pores.

In one embodiment of the present invention, the mixed matrix polymeric membrane comprises a plurality of at least a first metal-organic framework (MOF) and a polymeric matrix, wherein the plurality of MOFs are attached to the polymers of the polymeric matrix through a chemical bond such as a covalent bond. Other potential attachments can also be used such as hydrogen bonds or Van der Walls interactions. So while covalent bonding between the MOFs and polymers may be preferred, the MOFs could be attached to the polymers through covalent bonds, hydrogen bonds or Van der Walls interactions. Therefore, the use of “or” in the previous sentence is intended to encompass embodiments where the attachment of the MOFs to the polymeric matrix are through covalent bonds, hydrogen bonds, or Van der Walls interactions or through any combination thereof such as covalent bonds and hydrogen bonds, covalent bonds and Van der Walls interactions, or covalent bonds, hydrogen bonds, and Van der Walls interactions. Further, the membranes of the present invention can include a plurality of first, second, third, fourth, fifth, etc. MOFs such that a mixture of different MOFs can be used in a given membrane or such that a single type or species of MOF can be used in a given membrane. Non-limiting examples of specific types of MOFs and polymeric matrices or polymers are provided throughout this specification and incorporated into this section by reference. Particular examples of MOFs include isoreticular metal-organic framework-3 (IRMOF-3) and zeolitic imidazolate frameworks (ZIFs). Particular examples of polymers include polyimides such as 6-FDA-Durene or 6FDA-DAM. In embodiments where the MOFs are ZIFs, the covalent or hydrogen bonds or Van der Waals interaction may be formed between the polymeric matrix and a functional group added to the imidazolate ligand of the ZIFs. Non-limiting examples of specific types of functional groups are provided throughout this specification and incorporated into this section by reference. Particular examples include an amino group, an imine group, or combinations thereof. More than one functional group may be introduced into the MOFs. In particular instances, the MOFs are functionalized with at least 2, 3, 4, 5, 6, 7, 8, 9, or 10 functional groups. The pore size of the ZIFs can be tuned to the desired size by modifying the ratio of the imidazolate ligands and the functional groups introduced into the ZIFs. In some instances, the pore size will be in a range from 0.1 nm to 5 nm. In some instances, the pore size will be 0.1, 0.2, 0.3, 0.4, 0.5, 0.6, 0.7, 0.8, 0.9, 1, 1.5, 2, 2.5, 3, 2.5, 4, 4.5, or 5 nm. The two different ligands can be mixed in any ratio to synthesize hybrid ZIFs, and the ratio of functionalized ligand can be 1-99% mole percent of functional groups, depending on the desired selectivity of the membrane. Such membranes may include ZIFs comprising 60, 65, 70, 75, 80, 85, or 90 mole percent (i.e., mole fraction) of imidazolate ligands and 15, 20, 25, 30, or 35 mole percent of functional groups, depending on the desired selectivity of the membrane. In some instances, the ZIFs may comprise Zn, Cu, Co, or Fe, or any combination thereof and, in some instances, comprise a methyl imidazole carboxyaldehyde ligand, a methyl imidazole ligand, or a combination thereof. In particular instances, the plurality of ZIFs comprise ZIF-8-90. The plurality of MOFs may be comprised of all one MOF or different MOFs, or a combination of ZIFs and non-ZIF MOFs, and the mixed matrix membrane may comprise any appropriate concentration of MOFs, such as from 5% to 90% by mole. In some embodiments, the mixed matrix membranes can be void-free (i.e., the membranes do not include non-selective interfacial voids between the polymers of the membrane and the MOFs), substantially void-free (i.e., the size of the majority or all of the voids between the polymers of the membrane and the MOFs are less than or equal to five Angstroms in diameter), or do not have a “sieve-in-a-cage morphology. The membrane can be in the form of a thin film, flat sheet membrane, a spiral membrane, a tubular membrane, or a hollow fiber membrane. Additionally, the membranes disclosed herein have excellent permeability properties for a wide range of gases (e.g., N₂, H₂, CO₂, CH₄, C₂H₄, C₂H₆, C₃H₆, and C₃H₈) as well as selectivity performance (e.g., C₃H₆/C₃H₈, C₂H₄/C₂H₆, C₂H₆/C₃H₈, H₂/C₃H₈, H₂/N₂, H₂/C₃H₈, H₂/CH₄, CO₂/C₃H₈, CO₂/CH₄, CO₂/C₂H₄, N₂/CH₄, N₂/C₃H₈, CO₂/N₂). These permeability parameters can be further leveraged in that the faster or slower a gas moves through a particular membrane, the better selectivity can be created for a given pair of gases. Non-limiting examples of these permeability and selectivity properties of various membranes of the present invention are provided in the Examples, which are incorporated into this section by reference.

Also disclosed are processes of using the compositions and membranes disclosed throughout this specification. In one instance, the process can be used to separate two materials, gases, liquids, compounds, etc. from one another. Such a process can include contacting a mixture or composition having the materials to be separated on a first side of the composition or membrane, such that at least a first material is retained on the first side in the form of a retentate and at least a second material is permeated through the composition or membrane to a second side in the form of a permeate. In this sense, the composition or method could include opposing sides, wherein one side is the retentate side and the opposing side is the permeate side. The feed pressure of the mixture to the membrane or the pressure at which the mixture is feed to the membrane can range from 1, 2, 3, 4, 5, 6, 7, 8, 9, 10, 11, 12, 13, 14, or 15 atm or more or can range from 1 to 15 atm, 2 to 10 atm, or from 2 to 8 atm. Further the temperature during the separation step can range from 20, 25, 30, 35, 40, 45, 50, 55, 60, or 65° C. or more or from 20 to 65° C. or from 25 to 65° C. or from 20 to 30° C. The process can further include removing or isolating either or both of the retentate and/or the permeate from the composition or membrane. The retentate and/or the permeate can be subjected to further processing steps such as a further purification step (e.g., column chromatography, additional membrane separation steps, etc.). In particular instances, the process can be directed to removing at least one of N₂, H₂, CH₄, CO₂, C₂H₄, C₂H₆, C₃H₆, and/or C₃H₈ from a mixture. Examples of processes that the compositions and membranes disclosed herein can be used in include gas separation (GS) processes, vapour permeation (VP) processes, pervaporation (PV) processes, membrane distillation (MD) processes, membrane contactors (MC) processes, and carrier mediated processes, sorbent PSA (pressure swing absorption), etc. Further, it is contemplated that at least 2, 3, 4, 5, or more of the same or different membranes disclosed herein can be used in series with one another to further purify or isolate a targeted liquid, vapour, or gas material. Similarly, the membranes disclosed herein can be used in series with other currently known membranes to purify or isolate a targeted material.

In another aspect, there is disclosed a method of making the compositions or membranes disclosed throughout this specification. Such a method can include utilizing functionalized MOFs or functionalizing MOFs or ZIFs with at least one functional group, and attaching the functionalized MOFs or ZIFs with a polymer via a covalent or hydrogen bond or Van der Waals interaction. Non-limiting examples of specific types of functional groups are provided throughout this specification and incorporated into this section by reference.

Also disclosed in the context of the present invention are embodiments 1-37. Embodiment 1 includes a mixed matrix polymeric membrane comprising a polymeric matrix and a plurality of at least a first metal-organic framework (MOF), wherein the plurality of first MOFs are attached to the polymeric matrix through covalent or hydrogen bonds or Van der Waals interaction. Embodiment 2 is the mixed matrix polymeric membrane of embodiment 1, wherein the plurality of first MOFs are attached to the polymeric matrix through covalent bonds. Embodiment 3 is the mixed matrix polymeric membrane of embodiment 1, wherein the first MOF is a zeolitic imidazolate framework (ZIF). Embodiment 4 is the mixed matrix polymeric membrane of embodiment 3, wherein the attachment of the ZIFs to the polymeric matrix is formed between the polymeric matrix and a functional group on the imidazolate ligand of the ZIFs. Embodiment 5 is the mixed matrix polymeric membrane of any one of embodiments 3 or 4, wherein the ZIFs comprise a methyl imidazole carboxyaldehyde ligand, a methyl imidazole ligand, or a combination thereof. Embodiment 6 is the mixed matrix polymeric membrane of any one of embodiments 3 to 5, wherein the imidazolate ligands of the ZIFs are functionalized with at least two functional groups. Embodiment 7 is the mixed matrix polymeric membrane of embodiment 6, wherein the at least two functional groups are an amino group and an imine functional group. Embodiment 8 is the mixed matrix polymeric membrane of embodiment 3, wherein the ZIFs are ZIF-8-90. Embodiment 9 is the mixed matrix polymeric membrane of embodiment 1, wherein the first MOF is a isoreticular metal-organic framework-3 (IRMOF-3). Embodiment 10 is the mixed matrix polymeric membrane of any one of embodiments 1 to 9, further comprising a plurality of at least a second MOF that is different from the first MOF. Embodiment 11 is the mixed matrix polymeric membrane of embodiment 10, wherein the first MOF is a ZIF and the second MOF is IRMOF-3. Embodiment 12 is the mixed matrix polymeric membrane of embodiment 10, wherein the first MOF is a first ZIF, and the second MOF is a ZIF that is different from the first ZIF. Embodiment 13 is the mixed matrix polymeric membrane of any of embodiments 1-12, wherein the first or second MOFs comprise Zn, Cu, Co, or Fe, or any combination thereof. Embodiment 14 is the mixed matrix polymeric membrane of any of embodiments 1 to 13, wherein the membrane comprises at or between 1 to 99% functionalized imidazole. Embodiment 15 is the mixed matrix polymeric membrane of any of embodiments 1-14, wherein the first or second MOFs have a pore size between about 0.1 and 5 nm. Embodiment 16 is the mixed matrix polymeric membrane of any of embodiments 1-15, wherein the polymer comprises a polyimide. Embodiment 17 is the mixed matrix polymeric membrane of embodiment 16, wherein the polyimide comprises 6-FDA-Durene or 6FDA-DAM. Embodiment 18 is the mixed matrix polymeric membrane of any of embodiments 1-17, wherein the mixed matrix polymeric membrane comprises 5 to 90 mole percent of MOFs. Embodiment 19 is the mixed matrix polymeric membrane of any of embodiments 1-18, wherein the membrane is void-free or a majority of the voids in the membrane are 5 or less Angstroms in diameter. Embodiment 20 is the mixed matrix polymeric membrane of embodiment 19, wherein the membrane is substantially void free. Embodiment 21 is the mixed matrix polymeric membrane of any of embodiments 1-20, wherein the membrane is a flat sheet membrane, a spiral membrane, a tubular membrane, or a hollow fiber membrane. Embodiment 22 is the mixed matrix polymeric membrane of any one of embodiments 1 to 20, wherein the membrane is a thin film. Embodiment 23 is the mixed matrix polymeric membrane of any of embodiments 1-22, wherein the membrane is capable of separating a first gas from a second gas. Embodiment 24 is the mixed matrix polymeric membrane of embodiment 23, wherein the first gas is an olefin and the second gas is a paraffin. Embodiment 25 is a method for separating at least one component from a mixture of components, the process comprising: contacting a mixture of components on a first side of any one of the mixed matrix polymeric membranes of embodiments 1-24, such that at least a first component is retained on the first side in the form of a retentate and at least a second component is permeated through the membrane to a second side in the form of a permeate. Embodiment 26 is the method of embodiment 25, wherein the first component is a first gas and the second component is a second gas. Embodiment 27 is the method of embodiment 26, wherein the first gas is an olefin and the second gas is a paraffin. Embodiment 28 is the method of any one of embodiments 25 to 27, wherein the retentate and/or the permeate is subjected to a purification step. Embodiment 29 is the method of any one of embodiments 25 to 28, wherein the pressure at which the mixture is feed to the membrane is from 1 to 8 atm at a temperature ranging from 20° C. to 65° C. Embodiment 30 is a method of preparing the mixed matrix polymeric membrane of any one of embodiments 1-24 comprising: (a) functionalizing the at least first MOF with at least one functional group; and (b) attaching a plurality of the first MOFs to a polymer via a covalent or hydrogen bond or Van der Waals interaction. Embodiment 31 is the method of embodiment 30, wherein the plurality of first MOFs are attached to the polymeric matrix through covalent bonds. Embodiment 32 is the method of any one of embodiments 30 to 31, wherein the at least one functional group is an amino group or an imine functional group. Embodiment 33 is the method of any of embodiments 30 to 32, wherein the first MOF is a ZIF comprising at least two functional groups. Embodiment 34 is the method of embodiment 33, wherein the at least two functional groups are an amino group and an imine functional group. Embodiment 35 is the method of any of embodiments 30-34, wherein the polymer is a polyimide. Embodiment 36 is the method of embodiment 35, wherein the polyimide is 6-FDA-Durene or 6FDA-DAM. Embodiment 37 is the method of embodiment 36, wherein the permeate comprises gaseous H₂.

The terms “about,” “approximately,” and “substantially” are defined as being close to as understood by one of ordinary skill in the art, and in one non-limiting embodiment the terms are defined to be within 10%, preferably within 5%, more preferably within 1%, and most preferably within 0.5%.

The use of the word “a” or “an” when used in conjunction with the term “comprising” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The membranes, ingredients, components, compositions, or methods disclosed herein can “comprise,” “consist essentially of,” or “consist of” particular method steps, ingredients, components, compositions, etc. disclosed throughout the specification. With respect to the transitional phase “consisting essentially of,” in one non-limiting aspect, a basic and novel characteristic of the membranes disclosed herein are their improved selectivity parameters through the reduction of voids between the polymers and MOFs of the mixed matrix membranes.

Other objects, features and advantages disclosed herein will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIGS. 1A-B: (A) Schematic representation of an undesirable gap between the polymer matrix and the molecular sieve insert, commonly referred to as “sieve in a cage” morphology. (B) SEM of zeolite particles exhibiting “sieve-in-a-cage” morphology (Mahajan, et al., 2002).

FIG. 2: Preparation of hybrid ZIFs-based mixed matrix membranes.

FIG. 3: Linkers between polymers and ZIFs

FIG. 4: Synthesis of hybrid ZIF-8-90.

FIG. 5: Synthesis of ZIF-8-90-EDA.

FIG. 6: Synthesis of polyimide 6FDA-Durene.

FIG. 7: Preparation of hybrid ZIF-8-90-EDA/polyimide mixed matrix membrane.

FIG. 8: SEM images, XRD patterns and N2 isotherm of ZIF-8-90 and ZIF-8-90-EDA.

FIG. 9: FT-IR spectra of ZIF-90, ZIF-8-90 and ZIF-8-90-EDA.

FIG. 10: Pore size distribution of ZIF-8-90 and ZIF-8-90-EDA.

FIG. 11: FT-IR spectra of ZIF-8-90-EDA, polyimide 6FDA-Durene and mixed matrix membrane.

FIG. 12: SEM cross section image of mixed matrix membrane.

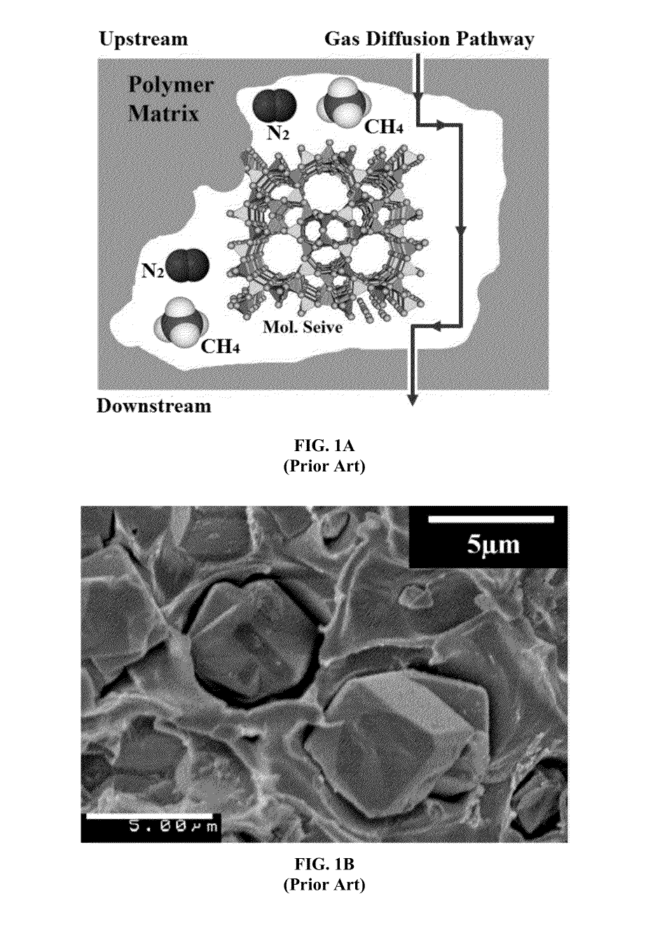

### DETAILED DESCRIPTION OF THE INVENTION

Currently available mixed matrix polymeric membranes that include MOFs do not have sufficient permeability/selectivity properties. This leads to performance and cost inefficiencies when using such membranes in applications such as gas separation applications.

As discussed above, the mixed matrix membranes of the present invention provide a solution to such performance issues. This solution is based on increasing the interaction between the MOFs and polymers such that the voids between the MOFs and polymers are reduced in size or number or both, thereby increasing the selectivity of the membranes. In preferred aspects, the interaction between the MOFs and polymers are increased through the formation of covalent bonds between the MOFs and polymers. However, and as discussed above, it is contemplated that other interactions such as hydrogen bonding or Van der Walls interactions can be used. Further, the pores of the MOFs can be modified as needed to further tune the selectivity of the membranes of the present invention (e.g., such tuning can be achieved by adjusting the ratio of ligands to functional groups within the MOFs).

The mixed matrix membranes can be used across a wide range of processes such as gas separation (GS) processes, vapour permeation (VP) processes, pervaporation (PV) processes, membrane distillation (MD) processes, membrane contactors (MC) processes, and carrier mediated processes.

These and other non-limiting aspects of the present invention are discussed in the following subsections.

### A. Metal-Organic Frameworks

MOFs are compounds having metal ions or clusters coordinated to organic molecules to form one-, two-, or three-dimensional structures that can be porous. By themselves, MOFs have been demonstrated to have very high gas sorption capacities, which suggest that gases generally will diffuse readily through MOFs if incorporated into a membrane. However, it has been discovered that MOFs attached to a polymeric membrane via a covalent or hydrogen bond or Van der Waals interaction create membranes that improve on the permeability and selectivity parameters by virtue of being void-free or substantially void-free, where either no voids or voids of less than several Angstroms are present at the interface of the polymers and the MOFs.

In general, it is possible to tune the properties of MOFs for specific applications using methods such as chemical or structural modifications. One approach for chemically modifying a MOF is to use a linker that has a pendant functional group for post-synthesis modification.

Any MOF either containing an appropriate functional group or that can be functionalized in the manner described herein can be used in the disclosed membranes. Examples include, but are not limited to, IRMOF-3, MOF-69A, MOF-69B, MOF-69C, MOF-70, MOF-71, MOF-73, MOF-74, MOF-75, MOF-76, MOF-77, MOF-78, MOF-79, MOF-80, DMOF-1-NH₂, UMCM-1-NH₂, and MOF-69-80 (Wang & Cohen, 2009; Rosi, et al., 2005).

In some embodiments, the MOFs are zeolitic imidazolate frameworks (ZIFs). ZIFs are a subclass or species of MOFs which have ordered porous structures with hybrid frameworks consisting of MN₄ (M=Co, Cu, Zn, etc.) clusters coordinated with organic imidazolate ligands (Banerjee, et al., 2008). Similar to other ordered porous materials like zeolites, the regular ZIF structure can be utilized in membrane related applications such as separations, membrane reactors, and chemical sensors. ZIFs have attractive properties such as high specific surface area, high stability, and chemically flexible framework that can be modified with functional groups by post-synthesis methods (Hayashi, et al., 2006; Park, et al., 2006; Venna, et al., 2010; Banerjee, et al., 2009; Morris, et al., 2008). Pure ZIF membranes have high performance at gas separation (Pan, et al., 2012a; Pan, et al., 2012b), but their applications are limited by high preparation cost. The synthesis and characterization of hybrid ZIFs with mixed linkers in the framework are described by Joshua A. Thompson (Thompson, et al., 2012). A description of one type of ZIFs and their use and preparation is described for example in US Patent Application No. 2010/0186588, International Patent Application No. WO 2007/0202038, International Patent Application No. WO 2008/140788, International Patent Application No. WO 2012/112122, International Patent Application No. WO 2012/159224, Zhang, et al., 2012, and Askari, et al., 2013, each of which are incorporated herein by reference in its entirety. For instance, a ZIF, can be synthesized by using solvothermal methods. Highly crystalline materials were obtained by combining the requisite hydrated metal salt (e.g., nitrate) and imidazole-type linker in an amide solvent such as N,N-diethylformamide (DEF). The resulting solutions were heated (85-150° C.) and zeolitic frameworks of the disclosure are precipitated after 48-96 h and readily isolated. In a further aspect, the imidazolate structures or derivatives can be further functionalized to impart functional groups that line the cages and channel, and particularly the pores to obtain a desired structure or pore size.

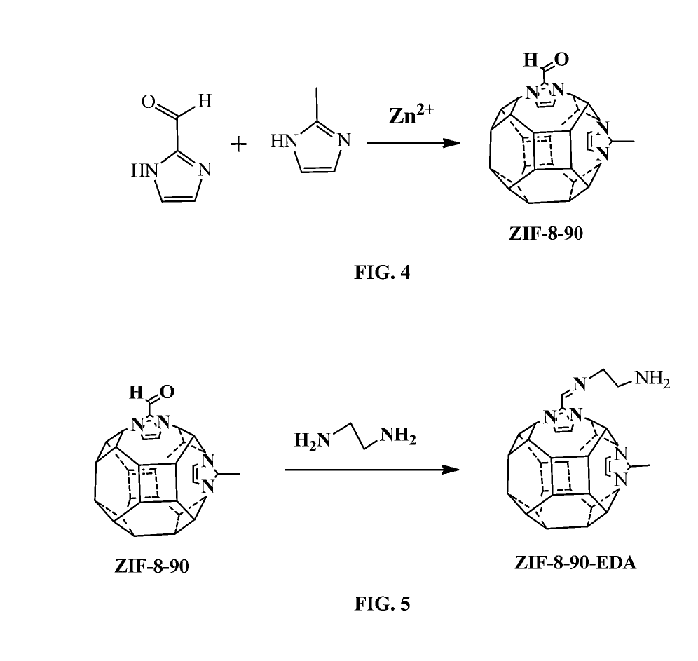

In some aspects, the hybridized zeolitic imidazolate frameworks are synthesized from zinc salts and mixed imidazole ligands. In particular aspects, the hybrid ZIF-8-90 is used. The hybrid ZIF-8-90 is synthesized via Zn(NO₃)₂.6H₂O co-ordinate with mixed ligands carboxaldehyde-2-imidazole and 2-methylimidazole, in which the carboxaldehyde group can react with amino compounds. FIG. 4 illustrates the synthesis of the hybrid ZIF-8-90, which has the following structure:

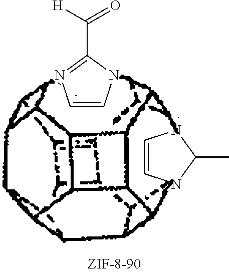

The ZIFs can be synthesized from the below imidazole ligands. At least one ligand can be functionalized. Non-limiting examples of such ligands that can be used to prepare ZIFs include:

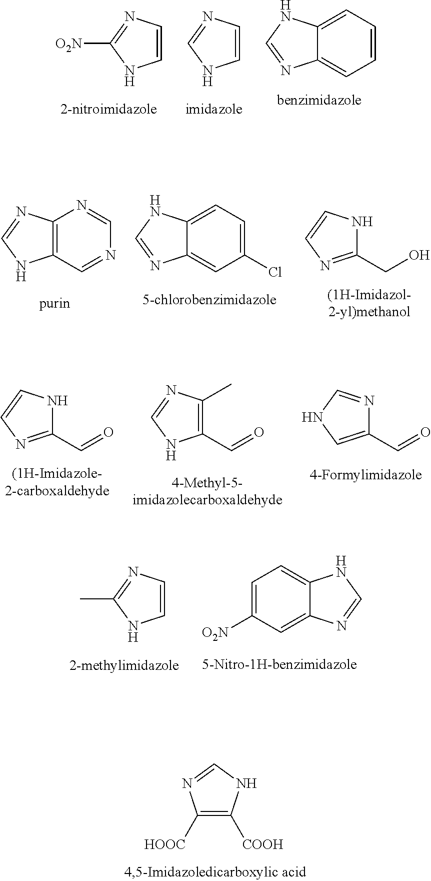

### B. Method of Making Membranes

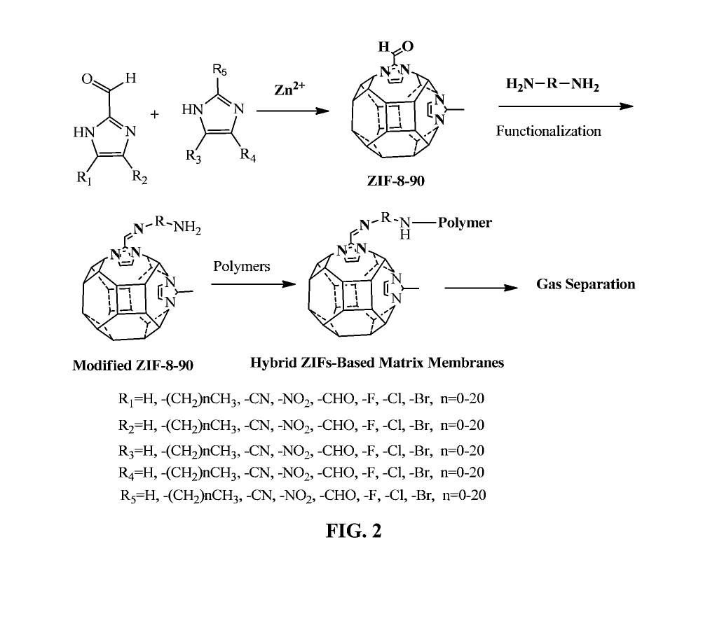

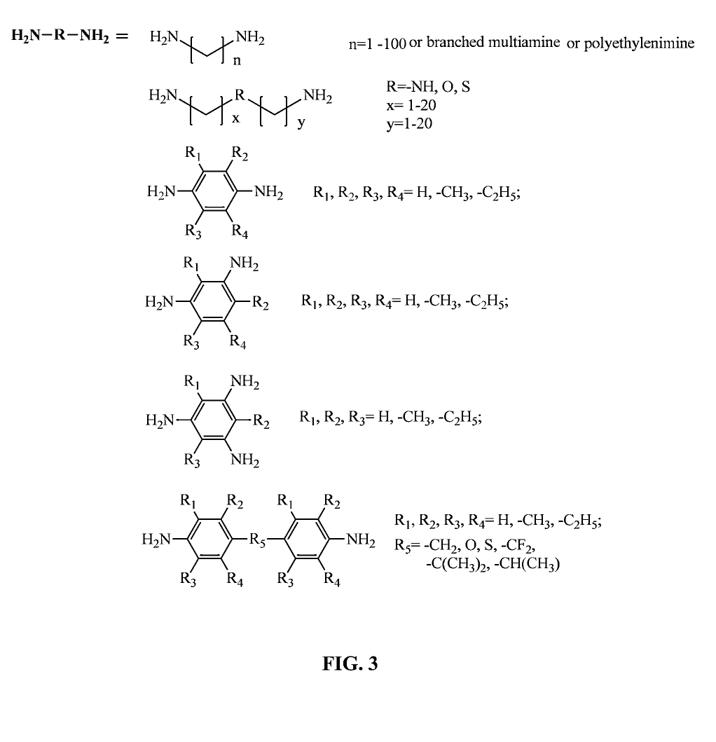

In some aspects, provided are methods of making the mixed matrix polymeric membranes disclosed herein. In some aspects, the methods involve functionalizing MOFs or ZIFs with at least one functional group and attaching the functionalized MOFs or ZIFs to a polymer via a covalent or hydrogen bond or Van der Waals interaction. FIGS. 2 and 3 provide a schematic overview of a non-limiting method of making such membranes. In FIG. 2, the imidazolate ligands are combined with the metal to create the ZIF, which is then functionalized. That modified ZIF is then attached to the polymer to create the polymeric membranes. In FIG. 2, the particular ZIF, ZIF-8-90, is employed in a non-limiting example; however, other imidazolate ligands and metals may be used to create a variety of ZIFs. FIG. 3 provides examples of various polymers that may be used.

The functional group can be any functional group or groups which allows attachment of the MOF to the polymeric membrane. In some embodiments, the MOF already contains the necessary functional group. In some embodiments, the MOF is modified to be functionalized. In some aspects, the MOF may be functionalized with more than one different functional group. For example, the functional group may be an amino group, an imine group, or combinations thereof.

The addition of the functional group to create modified ZIFs provides an avenue to tune the pore size of the modified ZIF. In particular, the pore size of the modified hybrid ZIFs is controlled by the ratio of the imidazole ligands to the introduced functional groups, and the pore sizes may be adjusted by changing the ratio of ligands on the MOFs and the functional groups. That ratio will affect the pore size of the ZIF, which may be between 0.1 and 5 nm. These pore sizes can be used to increase or tune the selectivity of the membrane for particular gases and other compounds in order to target the desired molecule or compound. In addition, the selection of the polymer for the membrane can also determine the selectivity of the membrane. In addition, the pore size changes not only with the ratio of ligands, but also with the kinds of ligands used. The pore size is controlled by two parameters: one is the ratio of ligands (unfunctionalized ligands to functionalized ligands), the other is the linker between the ZIF and the polymers. In the case of ZIF-8-90, the pore size increased with the increased ratio of carboxaldehyde-2-imidazole.

The MOFs disclosed herein are attached to the polymers of the membrane matrix via a hydrogen or covalent bond or Van der Waals interaction, either eliminating non-selective interfacial voids that are larger than the penetrating gas molecules between the polymers of the membrane and the MOF entirely (void-free) or reducing the size of the majority of or all of the voids present to less than several Angstroms (substantially void-free). The reduction or elimination of these voids effectively improves the selectivity of the membrane.

In addition to eliminating or reducing the size of voids, the materials and methods of making the disclosed membranes may allow for precise placement of a specified number of hybrid ZIFs layers in the membrane. Additionally, specific molecular interactions or direct covalent linking may be used to facilitate ordering or orientation of the hybrid ZIFs on the polymer or the membrane. Such methods also eliminate or reduce defects at the molecular sieve/polymer interface.

In addition to these steps, there are many known methods for making polymeric membranes from such compositions. Such methods that can be used include air casting (i.e., the dissolved polymer solution passes under a series of air flow ducts that control the evaporation of the solvents in a particular set period of time such as 24 to 48 hours), solvent or emersion casting, (i.e., the dissolved polymer is spread onto a moving belt and run through a bath or liquid in which the liquid within the bath exchanges with the solvent, thereby causing the formation of pores and the thus produced membrane is further dried), and thermal casting (i.e., heat is used to drive the solubility of the polymer in a given solvent system and the heated solution is then cast onto a moving belt and subjected to cooling).

### C. Polymers

The polymers of the mixed matrix membrane may be any polymers capable of bonding with a MOF via a functional group. Non-limiting examples of polymers that can be used in the context of the present invention include polyimide polymers. As noted above, the compositions and membranes can include a blend of any of these polymers (including blends of a single class of polymers and blends of different classes of polymers).

In some aspects, the polymer is a polyimide. Polyimide (PI) polymers are polymers of imide monomers. The general monomeric structure of an imide is:

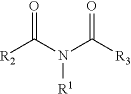

Polymers of imides generally take one of two forms: heterocyclic and linear forms. The structures of each are:

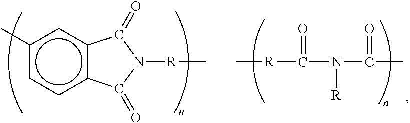

where R can be varied to create a wide range of usable PI polymers. Typically, n is greater than 1 or greater than 5 and typically from 10 to 10,000 or from 10 to 1000 or from 10 to 500. A non-limiting example of a specific PI (i.e., 6FDA-Durene) that can be used is described in the following reaction scheme:

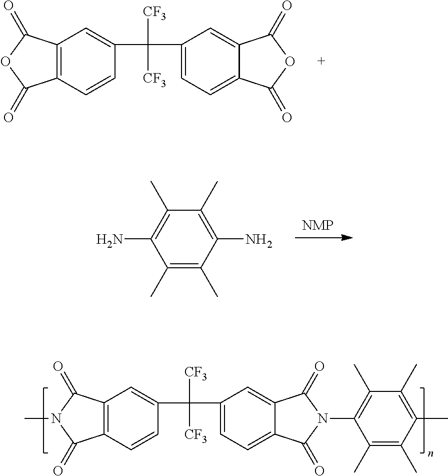

in which n is typically greater than 1 or greater than 5 and typically from 10 to 10,000 or from 10 to 1000 or from 10 to 500.

Additional PI polymers that can be used in the context of the present invention are described in U.S. Publication 2012/0276300, which is incorporated by reference. For instance, such polyimide polymers include both UV crosslinkable functional groups and pendent hydroxy functional groups: poly[3,3′,4,4′-benzophenonetetracarboxylic dianhydride-2,2-bis(3-amino-4-hydroxyphenyl)-hexafluoropropane] (poly(BTDA-APAF)), poly[4,4′-oxydiphthalic anhydride-2,2-bis(3-amino-4-hydroxyphenyl)-hexafluoropropane] (poly(ODPA-APAF)), poly(3,3′,4,4′-benzophenonetetracarboxylic dianhydride-3,3′-dihydroxy-4,4′-diamino-biphenyl) (poly(BTDA-HAB)), poly[3,3′,4,4′-diphenylsulfone tetracarboxylic dianhydride-2,2-bis(3-amino-4-hydroxyphenyl)-hexafluoropropane] (poly(DSDA-APAF)), poly(3,3′,4,4′-diphenylsulfone tetracarboxylic dianhydride-2,2-bis(3-amino-4-hydroxyphenyl)-hexafluoropropane-3,3′-dihydroxy-4,4′-diamino-biphenyl) (poly(DSDA-APAF-HAB)), poly[2,2′-bis-(3,4-dicarboxyphenyl) hexafluoropropane dianhydride-3,3′,4,4′-benzophenonetetracarboxylic dianhydride-2,2-bis(3-amino-4-hydroxyphenyl)-hexafluoropropane] (poly(6FDA-BTDA-APAF)), poly[4,4′-oxydiphthalic anhydride-2,2-bis(3-amino-4-hydroxyphenyl)-hexafluoropropane-3,3′-dihydroxy-4,4′-diamino-biphenyl] (poly(ODPA-APAF-HAB)), poly[3,3′,4,4′-benzophenonetetracarboxylic dianhydride-2,2-bis(3-amino-4-hydroxyphenyl)-hexafluoropropane-3,3′-dihydroxy-4,4′-diamino-biphenyl] (poly(BTDA-APAF-HAB)), and poly(4,4′-bisphenol A dianhydride-3,3′,4,4′-benzophenonetetracarboxylic dianhydride-2,2-bis(3-amino-4-hydroxyphenyl)-hexafluoropropane] (poly(BPADA-BTDA-APAF)). More generically, the PI polymers can have the following formula (I):

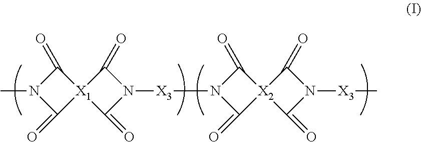

where the length of the polymer or “n” is typically greater than 1 or greater than 5 and typically from 10 to 10,000 or from 10 to 1000 or from 10 to 500, where —X1- of said formula (I) is

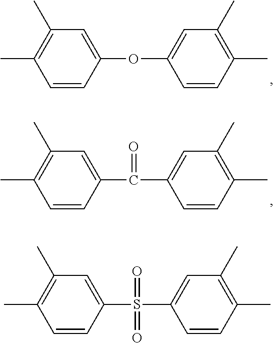

or mixtures thereof, —X2- of said formula (I) is either the same as —X1- or is selected from

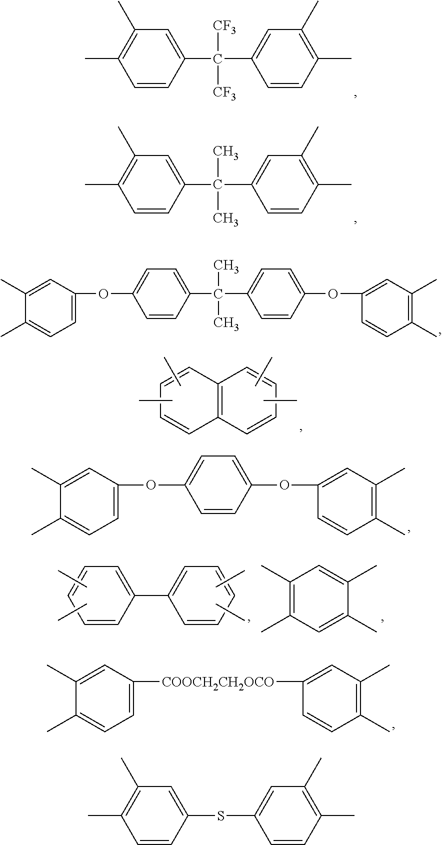

or mixtures thereof, —X3- of said formula (I) is

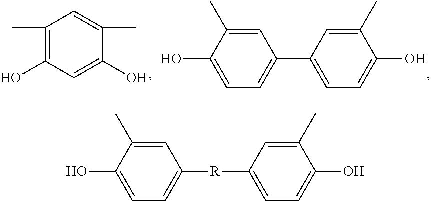

or mixtures thereof, —R— is

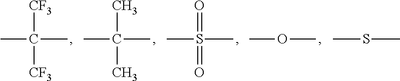

or mixtures thereof.

In some embodiments, the polymer is the polyimide 6FDA-FDA, which has the following structure:

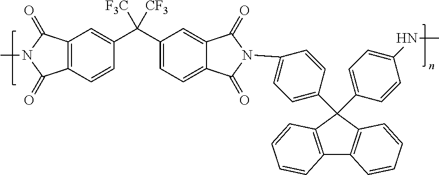

In some embodiments, the polymer is the polyimide 6FDA-DAM, which has the following structure:

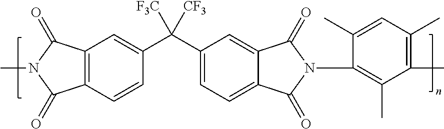

### D. Membrane Applications

The compositions and membranes disclosed herein have a wide-range of commercial applications. For instance, and with respect to the petro-chemical and chemical industries, there are numerous petro-chemical/chemical processes that supply pure or enriched gases such as He, N₂, and O₂, which use membranes to purify or enrich such gases. Further, removal, recapture, and reuse of gases such as CO₂ and H₂S from chemical process waste and from natural gas streams is of critical importance for complying with government regulations concerning the production of such gases as well as for environmental factors. Also, efficient separation of olefin and paraffin gases is key in the petrochemical industry. Such olefin/paraffin mixtures can originate from steam cracking units (e.g., ethylene production), catalytic cracking units (e.g., motor gasoline production), or dehydration of paraffins. Membranes of the invention can be used in each of these as well as other applications.

For instance, the compositions and membranes disclosed herein can be used in the purification, separation or adsorption of a particular species in the liquid or gas phase. In addition to separation of one or more gases from a gaseous mixture, the membranes can also be used to separate proteins or other thermally unstable compounds. The membranes may also be used in fermenters and bioreactors to transport gases into the reaction vessel and to transfer cell culture medium out of the vessel. Additionally, the membranes can be used to remove microorganisms from air or water streams, water purification, ethanol production in a continuous fermentation/membrane pervaporation system, and/or in detection or removal of trace compounds or metal salts in air or water streams.

In another instance, the compositions and membranes can be used in the separation of one or more liquids from liquid mixtures by pervaporation, such as in the removal of organic compounds (e.g., alcohols, phenols, chlorinated hydrocarbons, pyridines, ketones) from water such as aqueous effluents or process fluids. By way of example, a membrane that is ethanol-selective could be used to increase the ethanol concentration in relatively dilute ethanol solutions (e.g., less than 10% ethanol or less than 5% ethanol or from 5 to 10% ethanol) obtained by fermentation processes. A further liquid phase separation example that is contemplated with the compositions and membranes disclosed herein includes the deep desulfurization of gasoline and diesel fuels by a pervaporation membrane process (see, e.g., U.S. Pat. No. 7,048,846, which is incorporated by reference). Compositions and membranes disclosed herein that are selective to sulfur-containing molecules could be used to selectively remove sulfur-containing molecules from fluid catalytic cracking (FCC) and other naphtha hydrocarbon streams. Further, mixtures of organic compounds that can be separated with the compositions and membranes disclosed herein include ethylacetate-ethanol, diethylether-ethanol, acetic acid-ethanol, benzene-ethanol, chloroform-ethanol, chloroform-methanol, acetone-isopropylether, allylalcohol-allylether, allylalcohol-cyclohexane, butanol-butylacetate, butanol-1-butylether, ethanol-ethylbutylether, propylacetate-propanol, isopropylether-isopropanol, methanol-ethanol-isopropanol, and/or ethylacetate-ethanol-acetic acid.

In particular instances, the compositions and membranes disclosed herein can be used in gas separation processes in air purification, petrochemical, refinery, natural gas industries. Examples of such separations include separation of volatile organic compounds (such as toluene, xylene, and acetone) from chemical process waste streams and from flue gas streams. Further examples of such separations include the separation of CO₂ from natural gas, H₂ from N₂, CH₄, and Ar in ammonia purge gas streams, H₂ recovery in refineries, olefin/paraffin separations such as propylene/propane separation, iso/normal paraffin separations, and separation of H₂ from hydrocarbons such as H₂/C₂H₄/C₂H₆, and H₂/C₃H₆/C₃H₈, and the separation of a syngas mixture (H₂/CO₂/CO). Other industries that such membranes may be useful in include fermentation or agricultural industries.

Any given pair or group of gases that differ in molecular size, for example nitrogen and oxygen, carbon dioxide and methane, hydrogen and methane or carbon monoxide, helium and methane, can be separated using the blended polymeric membranes described herein. More than two gases can be removed from a third gas. For example, some of the gas components which can be selectively removed from a raw natural gas using the membranes described herein include carbon dioxide, oxygen, nitrogen, water vapor, hydrogen sulfide, helium, and other trace gases. Some of the gas components that can be selectively retained include hydrocarbon gases. In further instances, the membranes can be used on a mixture of gasses that include at least 2, 3, 4, or more gases such that a selected gas or gasses pass through the membrane (e.g., permeated gas or a mixture of permeated gases) while the remaining gas or gases do not pass through the membrane (e.g., retained gas or a mixture of retained gases).

Additionally, the compositions and membranes disclosed herein can be used to separate organic molecules from water (e.g., ethanol and/or phenol from water by pervaporation) and removal of metal (e.g., mercury(II) ion and radioactive cesium(I) ion) and other organic compounds (e.g., benzene and atrazene) from water).

A further use of the compositions and membranes disclosed herein include their use in chemical reactors to enhance the yield of equilibrium-limited reactions by selective removal of a specific product in an analogous fashion to the use of hydrophilic membranes to enhance esterification yield by the removal of water.

The compositions and membranes disclosed herein can also be fabricated into any convenient form such as sheets, tubes, spiral, or hollow fibers. They can also be fabricated into thin film composite membranes incorporating a selective thin layer comprising a UV-cross-linked PIM material and a porous supporting layer comprising a different polymer material.

Table 1 includes some particular non-limiting gas separation applications of the present invention.

TABLE 1

| Gas Separation | Application |
|---|---|
| O₂/N₂ | Nitrogen generation, oxygen enrichment |
| H₂/hydrocarbons | Refinery hydrocarbon recovery |
| H₂/CO | Syngas ratio adjustment |
| H₂/N₂ | Ammonia purge gas |
| CO₂/hydrocarbon | Acid gas treating, enhanced oil recovery, |
| | landfill gas upgrading, pollution control |
| H₂S/hydrocarbon | Sour gas treating |
| H₂O/hydrocarbon | Natural gas dehydration |
| H₂O/air | Air dehydration |
| Hydrocarbons/air | Pollution control, hydrocarbon recovery |
| Hydrocarbons from process | Organic solvent recovery, monomer |
| streams | recovery |
| Olefin/paraffin | Refinery |

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

### Synthesis of Hybrid ZIF-8-90

A solution of 100 mmol of Sodium formate, (100-x) mmol of 2-methylimidazole and x mmol of carboxaldehyde-2-imidazole in 250 mL of MeOH was prepared. To fully dissolve the OHC-IM ligand, the solution was heated to 50° C. until it became clear. A separate solution was prepared with 25 mmol of Zn(NO₃)₂.6H₂O and 250 mL of deionized H₂O. After the MeOH solution cooled to room temperature, the Zn salt solution was poured into the former solution and allowed to stir at room temperature for 2 h. The resulting milky precipitate was collected by centrifugation. And then the precipitate was dispersed in 100 mL of MeOH and washed three times. The powder was dried in an oven at 85° C. under vacuum for 48 h (FIG. 4).

### Example 2

### Synthesis of ZIF-8-90-EDA

2 ml of ethylenediamine and 2 g of hybrid ZIF-8-90 were mixed in 100 ml of methanol and refluxed over 24 h under N₂ atmosphere. The reaction mixture was cooled down to room temperature. The powder was collected by centrifugation and washed with methanol three times. The powder was dried in an oven at 85° C. under vacuum for 48 h (FIG. 5).

### Example 3

### Synthesis of Polyimide 6FDA-Durene

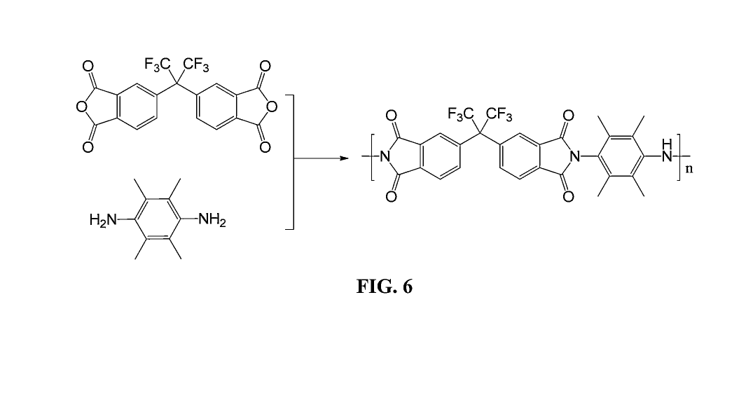

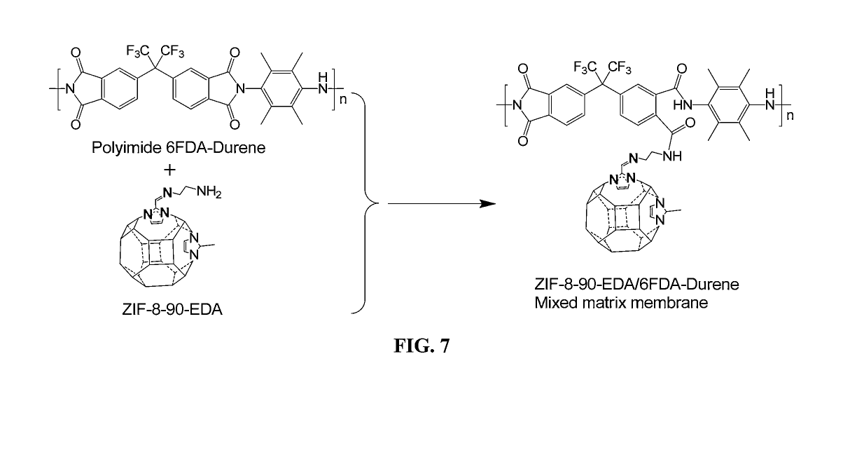

To a 250 mL of three-neck round flask, 4,4′-(Hexafluoroisopropylidene)diphthalic anhydride (10 mmol) and 2,3,5,6-Tetramethyl-p-phenylenediamine (10 mmol) was dissolved in 30 ml of anhydrous NMP and stirred for 24 h under N₂ atmosphere. Then the reaction solution was added 226.6 mmol acetic anhydride and 11.55 mmol of pyridine and stirred for 48 h. The polymer was precipitated from methanol three times. A white polymer was obtained and dried at 120° C. under vacuum for 48 h (FIG. 6).

### Example 4

### Preparation of Hybrid ZIF-8-90-EDA/Polyimide Mixed Matrix Membrane

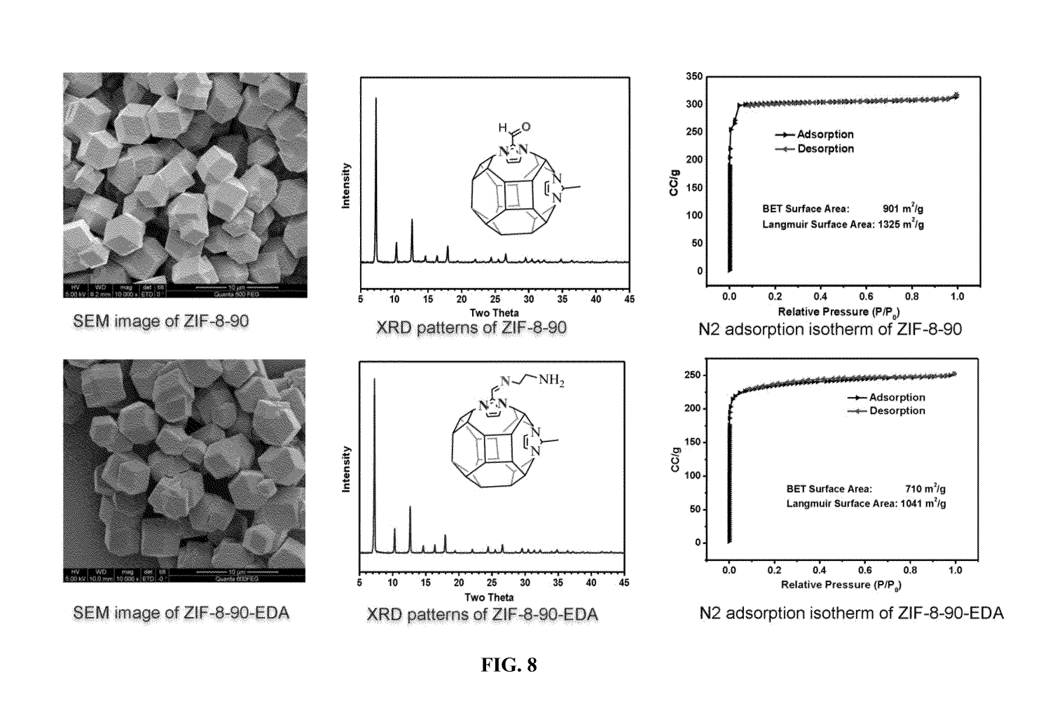

0.5 g of polyimide 6FDA-Durene was dissolved in 15 ml of CHCl₃. After being filtered by 0.25 μm film, the solution was mixed with 0.25 g of modified ZIF-8-90-EDA under sonication. The resulted mixture was cast in steel ring with glass plate and evaporated the solvent at room temperature. The resulted membrane was immersed in methanol overnight and then dried at 100° C. for 48 h under vacuum (FIG. 7).

### Example 5

### Morphology of Produced Mixed Matrix Membranes

Powder x-ray diffraction (XRD) patterns were recorded at room temperature on a Bruker D8 ADVANCE diffractometer in transmission geometry using CuKα radiation (λ=1.54059 Å) at 40 kV and 40 mA. Field-emission scanning electron microscope (SEM) pictures were taken by a FEI Quanta 600 FEG. Nitrogen physisorption isotherms were measured at 77 K on an automatic volumetric adsorption apparatus (Micromertics ASAP 2420). The samples were filled into glass ampoules and degassed in high vacuum at 393 K for 24 h before the start of the sorption measurements. Infrared spectra were acquired from KBr pellets of samples using a Nicolet 6700 FTIR spectrophotometer. Single gas permeation measurements were carried out using a custom-built gas permeameter. The permeameter consists of a stainless steel permeation cell which separates an upstream pressure transducer from a downstream pressure transducer. The permeation cell was tightly sealed and loaded into a constant volume permeation system. Vacuum was applied to both faces of the sample until the system leak rate minimizes. Before each gas was tested, 1-2 h of leak data is collected. Pure gas permeation measurements were performed at 35° C. and upstream pressure of ˜2 Bar.

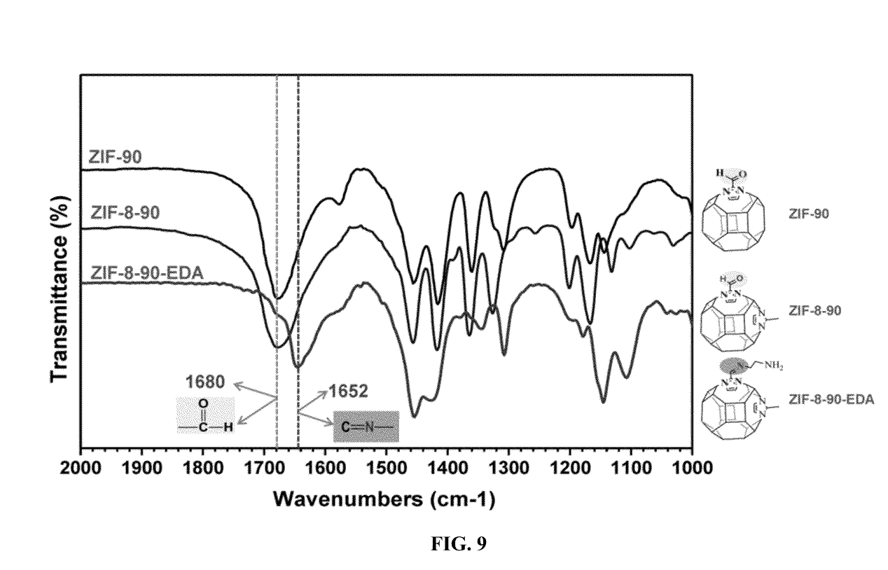

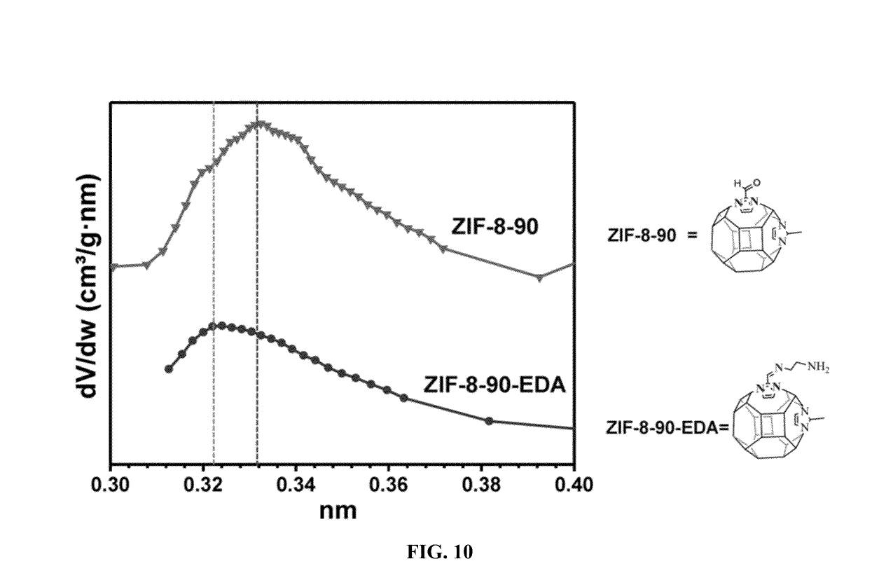

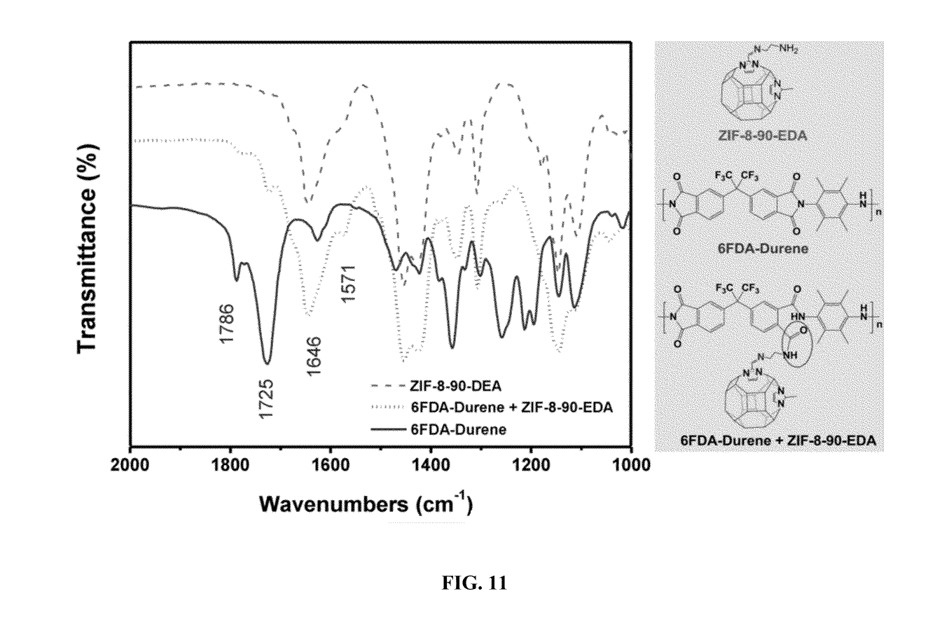

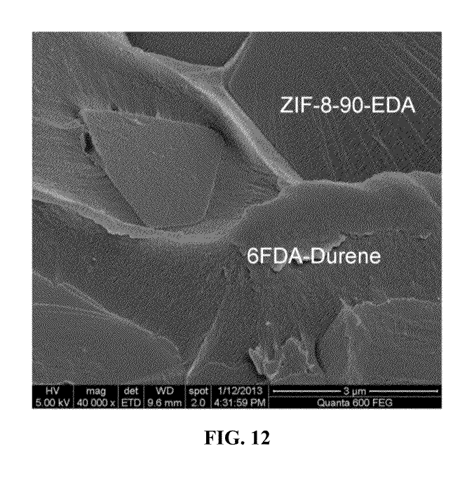

The results of SEM, XRD and BET indicate ZIF-8-90-EDA is crystalline and porous. FIG. 8. The characteristic peak 1680 cm⁻¹ attributed to asymmetric stretch of C═O in the aldehyde group in ZIF-90 and ZIF-8-90. When ZIF-8-90 reacted with ethylenediamine, the characteristic peak 1680 cm⁻¹ disappeared, and a new peak at 1652 cm⁻¹ appeared, which attributed to the characteristic peak of stretch of C═N of resulted ZIF-8-90-EDA. FIG. 9. When ZIF-8-90 reacted with ethylenediamine, the pore size of the resulted ZIF-8-90-EDA became smaller. FIG. 10. For polyimide 6FDA-Durene, after mixed with ZIF-8-90, the intensities of the characteristic peaks of imide groups at 1786 cm⁻¹ (asymmetric stretch of C═O in the imide group) and 1725 cm⁻¹ (symmetric stretch of C═O in the imide group) decreased and amide group at 1571 cm⁻¹ (stretch of C—N in the amide group) appeared. FIG. 11. From the SEM image, it can be seen that there is no void between the ZIF-8-90-EDA and polyimide (FIG. 12).

### Example 6

### Permeability and Selectivity Data

The gas transport properties were measured using the variable pressure (constant volume) method. Ultrahigh-purity gases (99.99%) were used for all experiments. The membrane is mounted in a permeation cell prior to degassing the whole apparatus. Permeant gas is then introduced on the upstream side, and the permeant pressure on the downstream side is monitored using a pressure transducer. From the known steady-state permeation rate, pressure difference across the membrane, permeable area and film thickness, the permeability coefficient is determined (pure gas tests). The permeability coefficient, P[cm³ (STP)·cm/cm²·s·cmHg], is determined by the following equation:

$$P = \frac{1}{760} \times \frac{V}{A} \times \frac{273}{273 + T} \times \frac{L}{760 p} \times \frac{d p}{d t}$$

where A is the membrane area (cm²), L is the membrane thickness (cm), p is the differential pressure between the upstream and the downstream (MPa), V is the downstream volume (cm³), R is the universal gas constant (6236.56 cm³·cmHg/mol·K), T is the cell temperature (° C.), and dp/dt is the permeation rate.

The gas permeabilities of polymer membranes are characterized by a mean permeability coefficient with units of Barrer. 1 Barrer=10⁻¹⁰ cm³ (STP)·cm/cm²·s·cmHg. The gas permeability coefficient can be explained on the basis of the solution-diffusion mechanism, which is represented by the following equation:

P=D×S

where D (cm²/s) is the diffusion coefficient; and S (cm³ (STP)/cm³·cmHg) is the solubility coefficient.

The diffusion coefficient was calculated by the time-lag method, represented by the following equation:

$$D = \frac{{L}^{2}}{6 θ}$$

where θ(s) is the time-lag. Once P and D were calculated, the apparent solubility coefficient S (cm³(STP)/cm³·cmHg) may be calculated by the following expression:

$$S = \frac{P}{D}$$

In gas separation, the membrane selectivity is used to compare the separating capacity of a membrane for 2 (or more) species. The membrane selectivity for one component (A) over another component (B) is given by the ratio of their permeabilities:

$${α}_{A / B} = \frac{{P}_{A}}{{P}_{B}}$$

Selectivity obtained from ratio of pure gas permeabilities is called the ideal membrane selectivity or the ideal permselectivity. This is an intrinsic property of the membrane material. The ideal selectivity of a dense membrane for gas A to gas B is defined as follows:

$$α = \frac{{P}_{A}}{{P}_{B}} = \frac{{D}_{A}}{{D}_{B}} * \frac{{S}_{A}}{{S}_{B}}$$

The gas permeability of ZIF-8-90-EDA-based mixed matrix membrane measured at 35° C., 2 Bar and comparison with polyimides is found in Tables 2 and 3.

TABLE 2

| | Membrane | | | | | | | | | |
|---|---|---|---|---|---|---|---|---|---|---|
| | Thickness | ZIF-8-90-EDA | Permeability (Barrer) | | | | | | | |
| Sample | (μm) | loading (wt %) | N₂ | CH₄ | H₂ | CO₂ | C₂H₄ | C₂H₆ | C₃H₆ | C₃H₈ |
| 6FDA-Durene | 50 | 0 | 26.89 | 24.89 | 364.41 | 473.11 | 42.07 | 14.33 | 38.23 | 3.57 |
| ZIF-8-90-EDA/6FDA-Durene | 94 | 33.33% | 318.48 | 278.62 | 3718.70 | 3907.10 | 654.67 | 202.58 | 278.19 | 22.49 |
| ZIF-8-90/6FDA-Durene | 96 | — | — | — | — | — | — | — | 295.97 | 27.19 |
| 6FDA-FDA | 63 | 0 | 3.72 | 3.00 | 126.64 | 88.77 | 6.67 | 2.61 | 6.31 | 0.36 |
| ZIF-8-90-EDA/6FDA-FDA | 75 | 33.33% | 7.12 | 5.53 | 236.37 | 202.27 | 9.75 | 3.31 | 7.18 | 0.37 |
| 6FDA-DAM | 70 | 0 | 66.00 | 60.40 | 901.21 | 1050.23 | 102.93 | 35.78 | 81.33 | 8.65 |
| ZIF-8-90-EDA/6FDA-DAM | 130 | 25% | 152.55 | 146.89 | 1797.04 | 2035.89 | 198.03 | 72.19 | 140.88 | 14.76 |

TABLE 3

| | | ZIF-8- | | | | | | | | | | | | | |
|---|---|---|---|---|---|---|---|---|---|---|---|---|---|---|---|
| | Membrane | 90-EDA | Ideal Selectivity | | | | | | | | | | | | |
| | Thickness | loading | C₃H₆/ | C₂H₄/ | C₂H₆/ | H₂/ | H₂/ | H₂/ | H₂/ | CO₂/ | CO₂/ | CO₂/ | N₂/ | | |
| Sample | (μm) | (wt %) | C₃H₈ | C₂H₆ | C₃H₈ | C₃H₈ | N₂ | C₃H₈ | CH₄ | C₃H₈ | CH₄ | C₂H₄ | CH₄ | N₂/C₃H₈ | CO₂/N₂ |
| 6FDA- | 50 | 0 | 10.72 | 2.94 | 4.02 | 102.16 | 13.55 | 102.16 | 14.64 | 132.63 | 19.01 | 11.25 | 1.08 | 7.54 | 17.59 |
| Durene | | | | | | | | | | | | | | | |
| ZIF-8-90- | 94 | 33.33% | 12.37 | 3.23 | 9.01 | 165.37 | 11.68 | 165.37 | 13.35 | 173.75 | 14.02 | 5.97 | 1.14 | 14.16 | 12.27 |
| EDA/6FDA- | | | | | | | | | | | | | | | |
| Durene | | | | | | | | | | | | | | | |
| ZIF-8- | 96 | — | 10.89 | — | — | — | — | — | — | — | — | — | — | — | — |
| 90/6FDA- | | | | | | | | | | | | | | | |
| Durene | | | | | | | | | | | | | | | |
| 6FDA-FDA | 63 | 0 | 17.56 | 2.56 | 7.26 | 351.78 | 34.05 | 352.49 | 42.19 | 247.08 | 29.57 | 13.31 | 1.24 | 10.35 | 23.87 |
| ZIF-8-90- | 75 | 33.33% | 19.41 | 2.94 | 8.95 | 638.84 | 33.20 | 638.83 | 42.77 | 546.68 | 36.60 | 20.75 | 1.29 | 19.24 | 28.41 |
| EDA/6FDA- | | | | | | | | | | | | | | | |
| FDA | | | | | | | | | | | | | | | |
| 6FDA-DAM | 70 | 0 | 9.41 | 2.88 | 4.14 | 104.19 | 13.65 | 104.24 | 14.92 | 121.48 | 17.39 | 10.20 | 1.09 | 7.63 | 15.91 |
| ZIF-8-90- | 130 | 25% | 9.54 | 2.74 | 4.89 | 121.75 | 11.78 | 121.72 | 12.23 | 137.90 | 13.86 | 10.28 | 1.04 | 10.33 | 13.35 |
| EDA/6FDA- | | | | | | | | | | | | | | | |
| DAM | | | | | | | | | | | | | | | |

### REFERENCES

The following references, to the extent that they provide exemplary procedural or other details supplementary to those set forth herein, are specifically incorporated herein by reference.
Aaron & Tsouris, Sep Sci Technol. 40:321-348, 2005.
Askari & Chung, Journal of Membrane Science. 444:173-183, 2013.
Banerjee, et al., J Am Chem Soc. 131:3875-3877, 2009.
Banerjee, et al., Science. 319:939-943, 2008.
Chung, et al., Prog Polym Sci. 32:483-507, 2007.
Hayashi, et al., Nature Mater. 6:501-506, 2007.
Hillock et al., Journal of Membrane Science. 314:193-199, 2008.
International Patent Application No. WO 2007/0202038.
International Patent Application No. WO 2008/140788.
International Patent Application No. WO 2012/112122.
International Patent Application No. WO 2012/159224.
Koros & Mahajan, J Membr Sci. 175:181, 2000.
Mahajan, et al., J Appl Polym Sci. 86:881, 2002.
Mersmann, et al., Chem Eng Technol. 23:937-944, 2000.
Moore, et al., AIChE J. 50:311, 2004.
Morris, et al., J J Am Chem Soc. 130:12626-12627, 2008
Pan, et al., Journal of Membrane Science. 390-391:93-98, 2012
Pan, et al., Journal of Membrane Science. 421-422:292-298, 2012b.
Park, et al., PNAS. 103:10186-10191, 2006.
Robeson, J Membr Sci. 320:390-400, 2008.
Robeson, J Membr Sci. 62:165, 1991.
Robeson, J Membr Sci. 62:165, 1991.
Rosi, et al., J Am Chem Soc. 127(5):1504-18, 2005.
Thompson, et al., Chem Mater. 24:1930, 2012.
U.S. Pat. No. 4,933,132
US Patent Application No. 2010/0186588.
Venna & Carreon, J Am Chem Soc. 132:76-78, 2010.
Wang & Cohen, Chem Soc Rev. 38(5):1315-29, 2009.
Zhang, et al., Journal of Membrane Science. 389:34-42, 2012.

1. A mixed matrix polymeric membrane comprising a polymeric matrix and a plurality of at least a first metal-organic framework (MOF), wherein the plurality of first MOFs are attached to the polymeric matrix through covalent or hydrogen bonds or Van der Waals interaction.

2. The mixed matrix polymeric membrane of claim 1, wherein the plurality of first MOFs are attached to the polymeric matrix through covalent bonds.

3. The mixed matrix polymeric membrane of claim 1, wherein the first MOF is a zeolitic imidazolate framework (ZIF).

4. The mixed matrix polymeric membrane of claim 3, wherein the attachment of the ZIFs to the polymeric matrix is formed between the polymeric matrix and a functional group on the imidazolate ligand of the ZIFs.

5. The mixed matrix polymeric membrane of claim 4, wherein the ZIFs comprise a methyl imidazole carboxyaldehyde ligand, a methyl imidazole ligand, or a combination thereof.

6. The mixed matrix polymeric membrane of claim 5, wherein the imidazolate ligands of the ZIFs are functionalized with at least two functional groups.

7. The mixed matrix polymeric membrane of claim 6, wherein the at least two functional groups are an amino group and an imine functional group.

8. The mixed matrix polymeric membrane of claim 3, wherein the ZIFs are ZIF-8-90.

9. The mixed matrix polymeric membrane of 1, wherein the first MOF is a isoreticular metal-organic framework-3 (IRMOF-3).

10. The mixed matrix polymeric membrane of claim 1, further comprising a plurality of at least a second MOF that is different from the first MOF.

11. The mixed matrix polymeric membrane of claim 10, wherein the first MOF is a ZIF and the second MOF is IRMOF-3.

12. The mixed matrix polymeric membrane of claim 10, wherein the first MOF is a first ZIF, and the second MOF is a ZIF that is different from the first ZIF.

13. The mixed matrix polymeric membrane of claim 1, wherein the first or second MOFs comprise Zn, Cu, Co, or Fe, or any combination thereof.

14. The mixed matrix polymeric membrane of claim 1, wherein the membrane comprises at or between 1 to 99% functionalized imidazole.

15. The mixed matrix polymeric membrane of claim 1, wherein the first or second MOFs have a pore size between about 0.1 and 5 nm.

16. The mixed matrix polymeric membrane of claim 1, wherein the polymer comprises a polyimide.

17. The mixed matrix polymeric membrane of claim 16, wherein the polyimide comprises 6-FDA-Durene or 6FDA-DAM.

18. The mixed matrix polymeric membrane of claim 1, wherein the mixed matrix polymeric membrane comprises 5 to 90 mole percent of MOFs.

19. The mixed matrix polymeric membrane of claim 1, wherein the membrane is void-free or a majority of the voids in the membrane are 5 or less Angstroms in diameter.

20. The mixed matrix polymeric membrane of claim 19, wherein the membrane is substantially void free.

21. The mixed matrix polymeric membrane of claim 1, wherein the membrane is a flat sheet membrane, a spiral membrane, a tubular membrane, or a hollow fiber membrane.

22. The mixed matrix polymeric membrane of claim 1, wherein the membrane is a thin film.

23. The mixed matrix polymeric membrane of claim 1, wherein the membrane is capable of separating a first gas from a second gas.

24. The mixed matrix polymeric membrane of claim 23, wherein the first gas is an olefin and the second gas is a paraffin.

25. A method for separating at least one component from a mixture of components, the process comprising: contacting a mixture of components on a first side of the mixed matrix polymeric membrane of claim 1, such that at least a first component is retained on the first side in the form of a retentate and at least a second component is permeated through the membrane to a second side in the form of a permeate.

26. A method of preparing the mixed matrix polymeric membrane of claim 1, comprising:
(a) functionalizing the at least first MOF with at least one functional group; and
(b) attaching a plurality of the first MOFs to a polymer via a covalent or hydrogen bond or Van der Waals interaction.
